(19) United States
(12) Patent Application Publication
(10) Pub. No.: US 2022/0313792 A1
(43) Pub. Date: Oct. 6, 2022

(54) HEMOGLOBIN-BASED NANOPARTICLES FOR OXYGEN DELIVERY

(71) Applicant: Ohio State Innovation Foundation, Columbus, OH (US)

(72) Inventors: Richard HICKEY, Columbus, OH (US); Andre PALMER, Columbus, OH (US)

(21) Appl. No.: 17/638,600

(22) PCT Filed: Aug. 28, 2020
(86) PCT No.: PCT/US2020/048367

§ 371 Date: Feb. 25, 2022

Related U.S. Application Data
(60) Provisional application No. 62/894,163, filed on Aug. 30, 2019.

(51) Int. Cl. A61K 38/42; A61K 38/38; A61K 9/00; A61K 9/14

(52) U.S. Cl. CPC A61K 38/42; A61K 38/385; A61K 9/0019; A61K 9/14

(57) ABSTRACT

Disclosed herein are oxygen transporting formulations, in particular those composed of hemoglobin-based nanoparticles, and their use and process of manufacture. These formulations are more uniform and monodisperse than prior hemoglobin-based oxygen carriers, such as polymeric hemoglobin. In addition, these formulations provide higher hemoglobin encapsulation efficiencies and higher hemoglobin content that hemoglobin-containing vesicles.

### CROSS-REFERENCE TO RELATED APPLICATIONS

This application claims the benefit of priority to U.S. Provisional Application No. 62/894,163, filed Aug. 30, 2019, the disclosure of which is incorporated herein by reference in its entirety.

### STATEMENT REGARDING FEDERALLY-SPONSORED RESEARCH

This disclosure was made with government support under Grant Nos. EB021926 awarded by the National Institute of Biomedical Imaging and Bioengineering and HL126945 awarded by the National Heart, Lung and Blood Institute, both of the National Institutes of Health. The government has certain rights in the invention.

### FIELD

The subject matter disclosed herein relates generally to oxygen transporting compositions and their methods of manufacture, more specifically formulations that contain a plurality of particles composed primarily of hemoglobin.

### BACKGROUND

The need for supplemental oxygen transport when red blood cells (RBCs) for transfusion are not available has long driven research into pharmaceutical alternatives. These materials have at times been called blood substitutes, artificial blood, and red blood cell substitutes. Because they typically aim to replace only the oxygen delivery capacity of RBCs, and not the various other functions of whole blood, they are often described as artificial oxygen carriers (AOCs).

A broadly proposed use for AOCs is to replace lost blood in emergency situations. In fact, the development of an AOC which subverts the classical constraints of blood transfusion products, such as type matching and storage concerns, is highly appealing. AOCs may be found to have even broader applications, such as use in bioreactors, ex vivo organ and tissue perfusion, or tumor oxygenation.

The most widely studied types of AOCs use hemoglobin (Hb) of biological origin because of its ready availability and highly efficient oxygen binding. Hemoglobin is the principal oxygen carrier of all mammals and is highly evolutionarily conserved (see Bunn, H. F., Blood, 1981, 58, 189-197). Hemoglobin can be readily purified from red blood cells, rendering it free of pathogens and blood type antigens while preserving its capacity for oxygen delivery. However, unmodified hemoglobin poses substantial risks and side effects if used directly, including hypertension, renal toxicity, and extravasation into tissue. These effects are largely due to the small size of hemoglobin compared to the red blood cell within which it normally resides (see Alayash, A. I. Trends Biotechnol. 2014, 32, 177-185). Investigation of hemoglobin-based oxygen carriers (HBOCs) spans decades of work and includes various attempts at engineering larger constructs to alleviate size-based effects while maintaining the capacity of oxygen delivery. HBOCs have been traditionally prepared by one or two routes: polymerization of hemoglobin (PolyHb) or encapsulation of hemoglobin into vesicles (HbV).

Polymeric hemoglobin solutions directly mix a chemical crosslinker with a hemoglobin solution to bind multiple protein molecules together, creating a mixture of various larger molecular weight polymers. The synthesis of polymeric hemoglobin necessarily creates a complex mixture due to non-specific binding of the chemical crosslinker to amino acids of the protein (see Simoni, J. et al. Anal. Chim. Acta 1993, 279, 73-88). To avoid side effects from low molecular weight polymeric hemoglobin fractions, various techniques have been used to isolate the desired size product. This necessitates the removal of products that are either too high or too low in size, reducing overall product yield and increasing processing time.

Encapsulating hemoglobin into vesicles represents another large group of technologies for making HBOCs. Hemoglobin-containing vesicles comprise a core-shell structure containing an aqueous core of high hemoglobin concentration and a membrane shell composed of lipids and/or polymers. These hemoglobin-containing vesicles vary in size, but can be produced in a preferred size range of 100-300 nm (see U.S. Pat. No. 7,417,118). For liposomal HbVs, the membrane materials are typically composed of specific lipids, a sterol, and polyethylene glycol conjugated lipid. Each of these species represents a cost beyond the active molecule (hemoglobin), as well as a decrease in the fraction of the particle made from hemoglobin. The encapsulation efficiency for HbVs is well under 50 percent.

Nanoparticles are extensively studied as carriers for small molecule drugs. Largely this is accomplished by adsorbing or otherwise incorporating a small molecule pharmaceutical onto an inert particle. A minority of these drug carrier systems use a polypeptide-based particle, typically albumin or gelatin. These systems rarely use the particle itself as the therapeutic agent. U.S. Pat. No. 5,069,936 describes a protein microsphere created through desolvation with the aid of a surfactant, with hemoglobin provided as a representative protein that could be used. Critically, the method described relied on the presence of a surfactant, particularly sodium dodecyl sulfate, whose addition is remarked as essential and without which the process only provides aggregates of uncontrollable size and shape.

U.S. Pat. No. 9,211,283 describes a human serum albumin (HSA) nanoparticle formulation for drug delivery. This formulation employs a desolvation technique on a protein solution, with the intended application of using the resulting nanoparticles as a carrier for a small molecule pharmaceutical agent, particularly photosensitizers.

There is a clear need for improved artificial oxygen carrier formulations based on hemoglobin with improved properties and consistent manufacturing.

### SUMMARY

Provided herein are compositions for oxygen transport that contain a plurality of particles composed primary of hemoglobin in addition to their methods of manufacture. These compositions are more uniform and monodisperse than prior hemoglobin-based oxygen carriers, such as polymeric hemoglobin. In addition, these compositions provide higher hemoglobin encapsulation efficiencies and higher hemoglobin content that hemoglobin-containing vesicles.

Thus in one aspect, an oxygen transporting formulation is provided comprising a plurality of particles;
wherein the plurality of particles has an average particle size of less than 1000 nm diameter;
wherein each particle within the plurality of particles comprises at least 25% by weight hemoglobin (based on the total weight of all proteins present in the particles) and optionally one or more additional proteins;
wherein each particle has an outer surface;

wherein the hemoglobin and/or other proteins present on the outer surface has been substantially crosslinked using a chemical crosslinker; and
wherein the oxygen transporting formulation is sufficiently free of surfactant.

In some embodiments, each particle is substantially composed of hemoglobin of human origin. In other embodiments, each particle is substantially composed of hemoglobin of bovine origin. In some embodiments, each particle is composed of a mixture of hemoglobin and human serum albumin (HSA).

In some embodiments, each particle within the plurality of particles comprises at least 25%, at least 30%, at least 40%, at least 50%, at least 75%, at least 80%, at least 90%, or more by weight hemoglobin based on the total weight of all proteins present in the particles. In some embodiments, each particle within the plurality of particles comprises at least 95% or more by weight hemoglobin based on the total weight of all proteins present in the particles.

In some embodiments, the plurality of particles is characterized in having an average particle size of less than 1000 nm, less than 900 nm, less than 800 nm, less than 700 nm, less than 600 nm, less than 500 nm, less than 500 nm, or less than 200 nm. In some embodiments, the plurality of particles is characterized in having an average particle size from 100 to 1000 nm, from 100 to 900 nm, from 100 to 800 nm, from 100 to 700 nm, from 100 to 600 nm, from 100 to 500 nm, from 100 to 400 nm, or from 100 to 300 nm.

In some embodiments, the plurality of particles is characterized in having a polydispersity index (PDI) from 0 to 0.3. In some embodiments, the plurality of particles is characterized in having a PDI from 0 to 0.1.

In some embodiments, the plurality of particles is characterized in having a zeta potential of less than −10 mV, for example less than −15 mV, less than −20 mV, less than −25 mV, less than −30 mV, or less than −35 mV. In some embodiments, the plurality of particles is characterized in having a zeta potential ranging from −40 mV to −10 mV, from −40 mV to −20 mV, from −40 mV to −30 mV, from −35 mV to −15 mV, from −35 mV to −20 mV, from −35 mV to −25 mV, or from −35 mV to −30 mV.

In some embodiments, the chemical crosslinker is an aldehyde-containing chemical crosslinker. In some embodiments, the chemical crosslinker is glutaraldehyde. In other embodiments, the chemical crosslinker is oxidized dextran.

In some embodiments, each particle within the plurality of particles has been further surface treated with one or more surface modulators. In some embodiments, the one or more surface modulators may be selected from human serum albumin (HSA), dextran, a polyelectrolyte, or one or more red blood cell membrane components.

In some embodiments, the oxygen transporting formulation further comprises at least one reducing agent. In some embodiments, the at least one reducing agent may be selected from N-acetyl-L-cysteine, ascorbic acid, uric acid, ergothioneine, methylene blue or derivates thereof such as dimethyl methylene blue, azure A, azure B, azure C, toluylene blue, brilliant cresyl blue, toluidine blue, or mixtures thereof.

In another aspect, a process is also provided for the synthesis of an oxygen transporting formulation as described herein, the process comprising:
(a) adding a desolvating agent to an aqueous solution of hemoglobin and optionally other proteins to provide a plurality of particles;
(b) adding a chemical crosslinker to substantially crosslink the hemoglobin and/or other optional proteins on the outer surface of each particle within the plurality of particles formed in step (a);
(c) isolating the plurality of particles provided from step (b) by substantially removing excess desolvating agent, chemical crosslinker, solvent, and other by-products; and
(d) resuspending the plurality of particles isolated in step (c) in a pharmaceutically acceptable carrier.

In some embodiments, the desolvating agent comprises a water-miscible polar solvent wherein which hemoglobin and/or the other optional proteins are insoluble. In some embodiments, the desolvating agent may be selected from ethanol, methanol, acetone, isopropyl alcohol, or combinations thereof.

In some embodiments, the process further comprises:
(b1) adding a reducing agent after addition of the chemical crosslinker in step (b) to further stabilizing the substantial crosslinking on the outer surface of each particle and deactivate any residual chemical crosslinker. In some embodiments, the reducing agent may be selected from sodium borohydride or sodium cyanoborohydride. In some embodiments, the reducing agent is added to reduce imine crosslink intermediates on the surface of the particle when an aldehyde-containing crosslinker is used such as glutaraldehyde or oxidized dextran. In some embodiments, the reducing agent is added to reduce imine intermediates in solution formed by the reaction of excess aldehyde-containing chemical crosslinker and an amine additive. In some embodiments, the amine additive may be selected from Tris buffer or glycine.

In some embodiments, the process comprises isolating the plurality of particles in step (c) by centrifugation or tangential flow filtration (TFF).

In some embodiments, the process comprises resuspending the plurality of particles in step (d) in an injectable solution suitable for clinical use.

Further provided are pharmaceutically acceptable oxygen transporting formulations comprising the plurality of particles described herein suspended in a pharmaceutically acceptable carrier optionally further containing pharmaceutically acceptable excipients. In some embodiments, the further pharmaceutically acceptable excipients may include any number of additives including, but not limited to, oncotic pressure agents, electrolytes, saccharides, amino acids, antioxidants, pH adjusters, and isotonizing agents.

### BRIEF DESCRIPTION OF DRAWINGS

FIG. 1 is a scheme illustrating the process for formation of hemoglobin-based nanoparticles by desolvation.

FIG. 2 is a scanning electron microscopy (SEM) image of the bovine hemoglobin derived nanoparticles formed using an oxidized dextran crosslinker as described in Example 1.

FIG. 3 is a scanning electron microscopy (SEM) image of 75% human hemoglobin/25% human serum albumin derived nanoparticles as described in Example 2.

FIG. 4 is a UV-visible absorption spectrum of the bovine hemoglobin derived nanoparticles described in Example 1.

FIG. 5 shows oxygen binding of the bovine hemoglobin derived nanoparticles described in Example 1.

FIG. 6 is a dynamic light scattering (DLS) histogram of the bovine hemoglobin derived nanoparticles described in Example 1.

FIG. 7 is a scheme illustrating the process for producing mixed 75% hemoglobin/25% human serum albumin derived nanoparticles as described in Example 2.

FIG. 8 is a scheme illustrating the process for producing HSA-coated hemoglobin nanoparticles as described in Example 4.

FIG. 9 is a scanning electron microscopy (SEM) image of hemoglobin derived nanoparticles formed using a glutaraldehyde crosslinker as described in Example 3.

FIG. 10 is a UV-visible absorption spectrum of the hemoglobin-derived nanoparticles crosslinked with glutaraldehyde as described in Example 3.

FIG. 11 shows a comparison of the zeta potential of the hemoglobin-derived nanoparticles crosslinked with glutaraldehyde (Hb-dNP), hemoglobin-derived nanoparticles crosslinked with oxidized dextran), the 75% Hb/25% HSA mixed nanoparticles (25% HSA), and the HAS-coated nanoparticles (HSA-coated) as described in Example 5.

FIG. 12 shows a comparison of the particle size of the hemoglobin-derived nanoparticles crosslinked with glutaraldehyde (Hb-dNP), hemoglobin-derived nanoparticles crosslinked with oxidized dextran), the 75% Hb/25% HSA mixed nanoparticles (25% HSA), and the HAS-coated nanoparticles (HSA-coated) as described in Example 5.

FIG. 13 shows a comparison of the deoxygenation stopped-flow kinetics of the hemoglobin nanoparticles (Hb-dNP), relaxed state polymerized hemoglobin (PolyHb-R), tense state polymerized hemoglobin (PolyHb-T), and red blood cells (RBC) as described in Example 6.

FIG. 14 are oxygen equilibrium curves for the hemoglobin nanoparticles (Hb-dNP), relaxed state polymerized hemoglobin (PolyHb-R), tense state polymerized hemoglobin (PolyHb-T), and red blood cells (RBC) as described in Example 6.

FIG. 15 is a representative tangential flow filtration set up that may be used in the purification of the nanoparticles described herein.

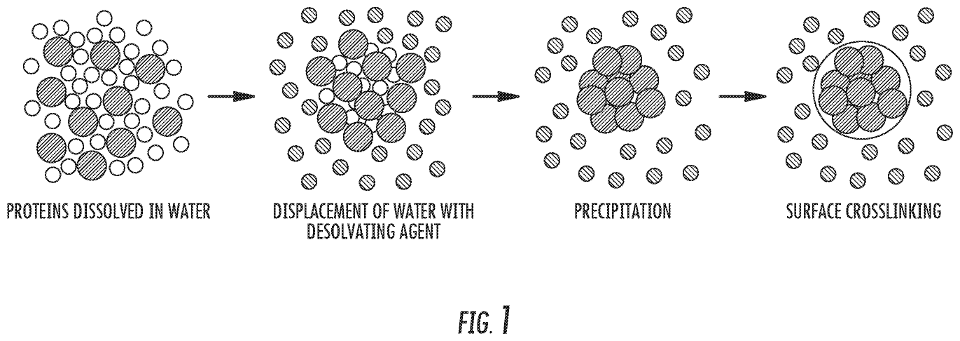

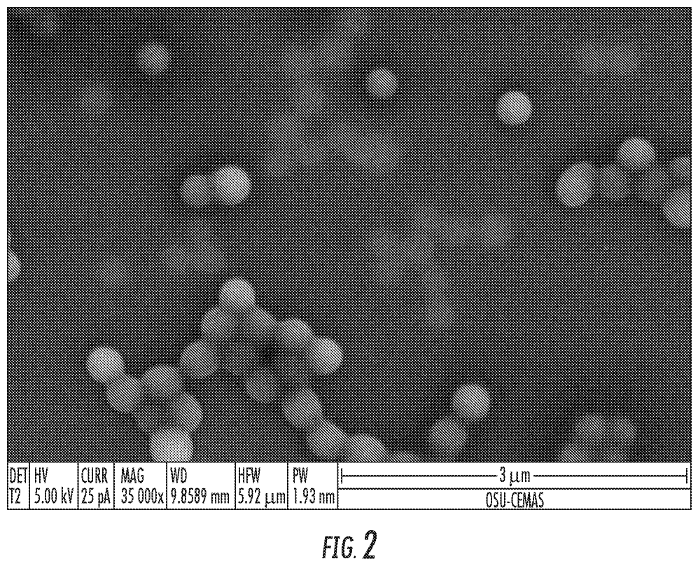

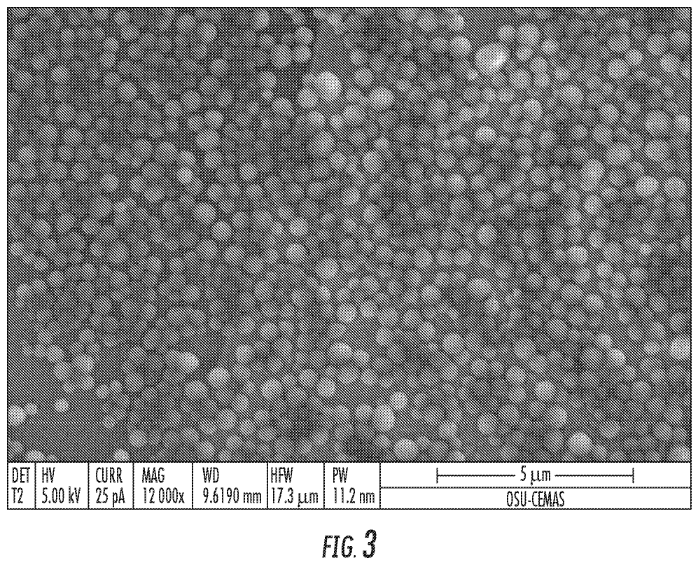

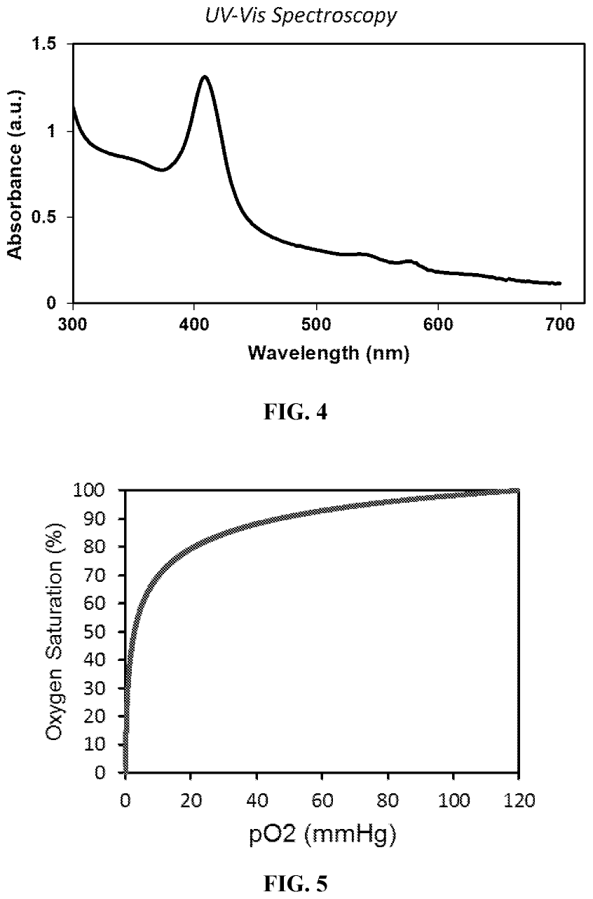

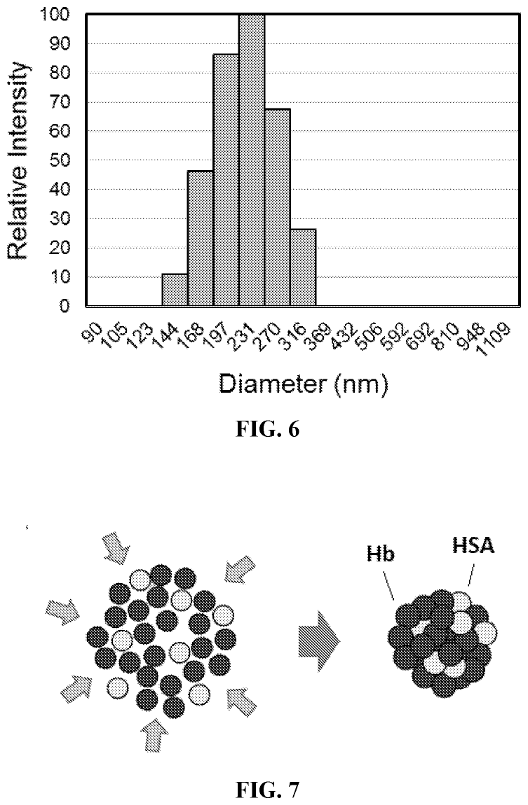

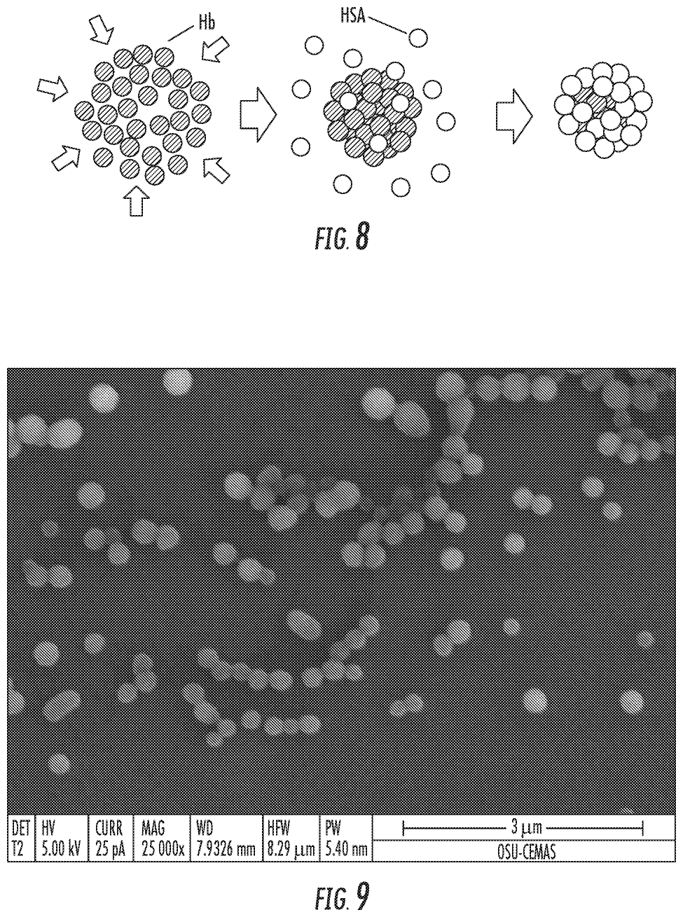

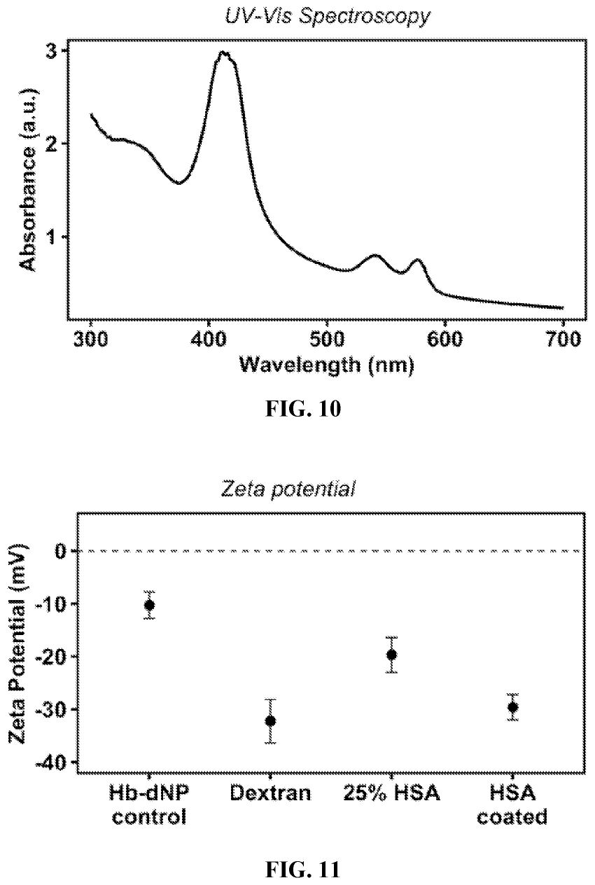

### DETAILED DESCRIPTION

The materials, compounds, compositions and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosure of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the specification and claims the word “comprise” and other forms of the word, such as “comprising” and “comprises”, means including but not limited to, and is not intended to exclude, for example, other additives, components, integers, or steps.

As used in the description and the appended claims, the singular forms “a”, “an”, and “the” include plural referents unless the context clearly dictates otherwise. Thus, for example, reference to “a composition” includes mixtures of two or more such compositions, and the like.

“Optional” or “optionally” means that the subsequently described feature, event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein from “about one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. Unless stated otherwise, the term “about” means within 5% (e.g. within 2% or 1%) of the particular value modified by the term “about”.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or in the case of an aerosol composition, gaseous.

A “pharmaceutically acceptable carrier” is a carrier, such as a solvent, suspending agent or vehicle, for delivering the disclosed materials to a patient. The carrier can be liquid or solid and is selected with the planned manner of administration in mind. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such medical and agents for pharmaceutical active substances is well known in the art. Except insofar as any conventional media or agent is incompatible with the active materials, its use in the therapeutic compositions is contemplated.

Reference will now be made in detail to specific aspects of the disclosed materials, compounds, compositions, articles, and methods, examples of which are illustrated in the accompanying Examples and Figures.

Provided herein are oxygen transporting formulations comprising:
a plurality of particles comprising one or more proteins;
wherein the plurality of particles have an average particle size of less than 1000 nm in diameter;
wherein each particle within the plurality of particles comprises at least 25 weight percent hemoglobin based on the total weight of all proteins present in the particles;
wherein each particle has an outer surface;
wherein the hemoglobin and or other protein molecule present on the outer surface have been substantially crosslinked using a crosslinker; and
wherein the oxygen transporting formulation is sufficiently free of surfactant.

Provided herein are oxygen transporting formulations comprising a plurality of particles;
wherein the plurality of particles has an average particle size of less than 1000 nm in diameter;
wherein each particle within the plurality of particles comprises at least 25 weight percent hemoglobin and optionally one or more additional proteins;
wherein each particle has an outer surface;
wherein the hemoglobin and/or protein molecules present on the outer surface have been substantially crosslinked using a chemical crosslinker; and
wherein the oxygen transporting formulation is sufficiently free of surfactant.

In some embodiments, each particle comprises at least 25% by weight hemoglobin and/or optionally other proteins the total protein weight of the particle. In some embodiments, each particle comprises at least 30% by weight hemoglobin and/or optionally other proteins the total protein weight of the particle. In some embodiments, each particle comprises at least 35% by weight hemoglobin and/or optionally other proteins the total protein weight of the particle. In some embodiments, each particle comprises at least 40% by weight hemoglobin and/or optionally other proteins the total protein weight of the particle. In some embodiments, each particle comprises at least 50% by weight hemoglobin and/or optionally other proteins the total protein weight of the particle. In some embodiments, each particle comprises at least 60% by weight hemoglobin and/or optionally other proteins the total protein weight of the particle. In some embodiments, each particle comprises at least 70% by weight hemoglobin and/or optionally other proteins the total protein weight of the particle. In some embodiments, each particle comprises at least 75% by weight hemoglobin and/or optionally other proteins the total protein weight of the particle. In some embodiments, each particle comprises at least 80% by weight hemoglobin and/or optionally other proteins the total protein weight of the particle. In some embodiments, each particle comprises at least 90% by weight hemoglobin and/or optionally other proteins the total protein weight of the particle. In some embodiments, each particle comprises at least 95% by weight hemoglobin and/or optionally other proteins based on the total protein weight of the particle.

In some embodiments, each particle comprises 25% hemoglobin and 75% human serum albumin (HSA) for the protein component of the particle. In some embodiments, each particle comprises 50% hemoglobin and 50% HSA for the protein component of the particle. In some embodiments, each particle comprises 75% hemoglobin and 25% HSA for the protein component of the particle. In some embodiments, each particle comprises 95% or more hemoglobin for the protein component of the particle. In some embodiment, each particle comprises 100% hemoglobin for the protein component of the particle.

In some embodiments, the plurality of particles is characterized in having an average particle size, as determined by dynamic light scattering, of less than 1000 nm. In some embodiments, the plurality of particles is characterized in having an average particle size of less than 900 nm. In some embodiments, the plurality of particles is characterized in having an average particle size of less than 800 nm. In some embodiments, the plurality of particles is characterized in having an average particle size of less than 700 nm. In some embodiments, the plurality of particles is characterized in having an average particle size of less than 600 nm. In some embodiments, the plurality of particles is characterized in having an average particle size of less than 500 nm. In some embodiments, the plurality of particles is characterized in having an average particle size of less than 400 nm. In some embodiments, the plurality of particles is characterized in having an average particle size of less than 300 nm. In some embodiments, the plurality of particles is characterized in having an average particle size of less than 200 nm.

In some embodiments, the plurality of particles is characterized in having an average particle size, as determined by dynamic light scattering, from 100 to 1000 nm. In some embodiments, the plurality of particles is characterized in having an average particle size from 100 to 900 nm. In some embodiments, the plurality of particles is characterized in having an average particle size from 100 to 800 nm. In some embodiments, the plurality of particles is characterized in having an average particle size from 100 to 700 nm. In some embodiments, the plurality of particles is characterized in having an average particle size from 100 to 600 nm. In some embodiments, the plurality of particles is characterized in having an average particle size from 100 to 500 nm. In some embodiments, the plurality of particles is characterized in having an average particle size from 100 to 400 nm. In some embodiments, the plurality of particles is characterized in having an average particle size from 100 to 300 nm. In some embodiments, the plurality of particles is characterized in having an average particle size from 100 to 200 nm.

In some embodiments, the plurality of particles is characterized in having a polydispersity index from about 0 to 0.3. In some embodiments, the plurality of particles is characterized in having a polydispersity index from 0 to 0.25. In some embodiments, the plurality of particles is characterized in having a polydispersity index of 0 to 0.2. In some embodiments, the plurality of particles is characterized in having a polydispersity index of 0 to 0.15. In some embodiments, the plurality of particles is characterized in having a polydispersity index of 0 to 0.1. In some embodiments, the plurality of particles is characterized in having a polydispersity index less than 0.3. In some embodiments, the plurality of particles is characterized in having a polydispersity index less than 0.1.

In some embodiments, the plurality of particles is characterized in having a zeta potential of less than −10 mV. In some embodiments, the plurality of particles is characterized in having a zeta potential of less than −15 mV. In some embodiments, the plurality of particles is characterized in having a zeta potential of less than −20 mV. In some embodiments, the plurality of particles is characterized in having a zeta potential of less than −25 mV. In some embodiments, the plurality of particles is characterized in having a zeta potential of less than −30 mV. In some embodiments, the plurality of particles is characterized in having a zeta potential of less than −35 mV. In some embodiments, the plurality of particles is characterized in having a zeta potential of less than −10 mV, less than −12 mV, less than −14 mV, less than −16 my, less than −18 mV, less than −20 mV, less than −22 mV, less than −24 mV, less than −26 mV, less than −28 mV, less than −30 mV, less than −32 mV, less than −34 mV, less than −36 mV, or less than −38 mV.

In some embodiments, the plurality of particles is characterized in having a negative zeta potential. In some embodiments, the plurality of particles is characterized in having a zeta potential ranging from −40 mV to −10 mV. In some embodiments, the plurality of particles is characterized in having a zeta potential ranging from −40 mV to −20 mV. In some embodiments, the plurality of particles is characterized in having a zeta potential ranging from −40 mV to −30 mV. In some embodiments, the plurality of particles is characterized in having a zeta potential ranging from −35 mV to −15 mV. In some embodiments, the plurality of particles is characterized in having a zeta potential ranging from −35 mV to −20 mV. In some embodiments, the plurality of particles is characterized in having a zeta potential ranging from −35 mV to −25 mV. In some embodiments, the plurality of particles is characterized in having a zeta potential ranging from −35 mV to −30 mV. In some embodiments, the plurality of particles is characterized in having a zeta potential ranging from −30 mV to −10 mV. In some embodiments, the plurality of particles is characterized in having a zeta potential ranging from −30 mV to −15 mV. In some embodiments, the plurality of particles is characterized in having a zeta potential ranging from −30 mV to −20 mV. In some embodiments, the plurality of particles is characterized in having a zeta potential ranging from −30 mV to −25 mV. In some embodiments, the plurality of particles is characterized in having a zeta potential ranging from −25 mV to −10 mV. In some embodiments, the plurality of particles is characterized in having a zeta potential ranging from −25 mV to −15 mV. In some embodiments, the plurality of particles is characterized in having a zeta potential ranging from −25 mV to −20 mV. In some embodiments, the plurality of particles is characterized in having a zeta potential ranging from −20 mV to −10 mV. In some embodiments, the plurality of particles is characterized in having a zeta potential ranging from −20 mV to −15 mV. In some embodiments, the plurality of particles is characterized in having a zeta potential ranging from −15 mV to −10 mV.

Hemoglobin as used in the present disclosure may be isolated directly from red blood cells or may be recombinant. Hemoglobin may be readily purified from fresh or stored blood using methods known to those of skill in the art. In an alternative embodiment, hemoglobin may be purchased as a reagent, but this is not preferred due to the potential presence of metHb as well as lower oxygen carrying capacity. For example, hemoglobin for use in the present disclosure may be prepared by removing stroma from a human erythrocyte and adjusting the pH of the hemoglobin solution to 7.0 to 7.5. The pH of the hemoglobin solution may be adjusted using an aqueous solution of sodium hydroxide, sodium carbonate, or sodium hydrogen carbonate or by adding a buffer such as Tris, bis-Tris, HEPES, or a buffer of inorganic phosphates.

To form the particles described herein, hemoglobin is dissolved in an aqueous buffer to which a desolvating agent is added. The desolvating agent is a liquid in which hemoglobin is poorly soluble but that is miscible with water. Polar solvents such as alcohols are often well suited to be desolvating agents in this process. As the desolvating agent is added, solute-solute interactions dominated over solute solvent forces, driving nucleating of hemoglobin precipitates. With appropriate conditions, nucleation results in the rapid formation of particles.

Suitable desolvating agents include alcohol desolvating agents, such as methanol, ethanol, propanols, butanols, or mixtures thereof, or acetone. In some embodiments, the addition of concentrated polyethylene glycol solutions (>40% in water) can be used to effect desolvation.

Upon formation of particles, a chemical crosslinker is added to stabilize the particles and fix their size and shape. A suitable chemical crosslinker is used to bind several sites across the surface of the particle, halting particle growth and limiting particle-particle interaction. Depending upon the chemical crosslinker used, it may be prudent to chemically deactivate excess reactants with a suitable quenching agent.

Examples of suitable chemical crosslinkers include polyfunctional agents that will crosslink proteins, for example glutaraldehyde, succindialdehyde, activated forms of polyoxyethylene and dextran, α-hydroxy aldehydes, such as glycoaldehyde, N-maleimido-6-aminocaproyl(2′-nitro-4′-sulfonic acid)phenyl ester, m-maleimidobenzoic acid N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl (4-iodoacetyl)aminobenzoate, sulfosuccinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class, or the aryl dihalide class, among others. When the chemical crosslinker used is not an aldehyde, the nanoparticles formed are stable.

When the chemical crosslinker is an aldehyde, the nanoparticles formed are not stable until mixed with a suitable reducing agent to reduce the less stable bonds to form more stable bonds. Examples of suitable reducing agents include sodium borohydride, sodium cyanoborohydride, sodium dithionite, trimethylamine, t-butylamine, morpholine borane, and pyridine borane. In some embodiments, the reducing agent is also used to reduce reactivity of any residual chemical crosslinker present in the solution to prevent crosslinking between particles.

Any time after precipitation, the surfaces of the particles may be modified with the addition of a surface treatment agent. Depending upon solubility and function of the surface treatment agent in the reaction buffer, it may be prudent to perform the surface modification after removal of the desolvating agent.

Representative examples of surface treatment agents include, but are not limited to: other proteins such as human serum albumin (HSA); oligosaccharides; polysaccharides, such as, for example dextran; a polyelectrolyte such as an ionomer, lignosulfonates, sulfonated tetrafluoroethylene polymers, poly(3,4-ethylene dioxythiophene) polystyrene sulfonate, polyacrylamide, polyacrylic acid, polyallylamine hydrochloride, poly(2-acrylamido-2-methyl-1-propanesulfonic acid), polyaniline, poly(acrylamido-N-propyltrimethylammonium chloride), poly [(3-methylacryloylamino)-propyl]trimethylammonium chloride), polyaspartic acid, polypyridinium salts, polystyrene sulfonate, and sodium polyaspartate; red blood cell membrane components such as red blood cell membrane lipids including cholesterol, phosphatidylcholine, sphingomyelin, phosphatidylethanolamine, phosphoinositol, and phosphatidyl serine or red blood cell membrane proteins such as Band 3, Aquaporin 1, Glutl, Kidd antigen protein, RhAg, Na⁺/K⁺—ATPase, Ca²⁺—ATPase, Na⁺K⁺ 2Cl⁻ cotransporter, Na⁺ Cl⁻ cotransporter, Na—H exchanger, K-Cl cotransporter, Gardos channel, ICAM-4, BCAM, Protein 4.1R, Glycophorin C and D, XK, RhD/RhCE, Duffy protein, Adducin, Dematin, flotillins, stomatins (band 7), G-proteins, and β-andrenergic receptors; polyethyleneglycols; and zwitterionic polymers.

The desolvating agent may be removed through a variety of means of buffer exchange known to those skilled in protein or particle modification. Small volumes may be washed via ultracentrifugation, but these techniques do not translate well to clinically meaningful scales of production. Larger batches (e.g. greater than 10 mL) may be washed into fresh buffer quite effectively by means of tangential flow filtration (TFF). A hollow-fiber TFF cartridge with polysulfone membrane pore size of about 50 nm is effective for rapid buffer exchange while retaining particles in the system reservoir. Such a TFF system may also be used concentrate materials to a desired concentration (measured in particle/mL or mg Hb/mL). In some embodiments, particle compositions used for clinical applications must be free of any toxic or contaminating material prior to use. Therefore, the particles described herein can be sterilized by any of the different known means in the art such as autoclaving, ethylene oxide, gamma-irradiation, or sterile filtration using membranes of a known size. In some embodiments, the particles described herein are prepared under completely sterile conditions.

The particles of the present disclosure may be dehydrated for improved stability on storage. The preferred method of dehydration is freeze-drying or lyophilization. Optionally, a lyoprotectant may be used as an additive to improve the stability during the freeze-drying and during reconstitution in an aqueous medium (see Anhorn, M. G. et al. Int. J. Pharm. 2008, 363, 162-169).

In some embodiments, the particles described herein are formulated as an oxygen transporting formulation suitable for clinical applications. In such oxygen transporting formulations, the plurality of particles described herein are typically dispersed in a pharmaceutically acceptable carrier or diluent. In some embodiments, the pharmaceutically acceptable carrier or diluent is water. The oxygen transporting formulation may further comprise any number of additional additives such as oncotic pressure agents, electrolytes, saccharides, amino acids, antioxidants, pH adjusters, and isotonizing agents.

Oncotic pressure agents include various polymers that may be used for medical purposes, including but not limited to: a dextran such as a low molecular weight dextran; a dextran derivative such as carboxymethyl dextran, carboxydextran, cationic dextran, or dextran sulfate; hydroxyethyl starch; hydroxypropyl starch; gelatin such as modified gelatin; albumin such as from human plasma, human serum albumin, heated human plasma protein, and recombinant serum albumin; polyethylene glycol; polyvinyl pyrrolidinone; carboxymethyl cellulose; acacia gum; glucose; a dextrose such as glucose monohydrate; oligosaccharides; a polysaccharide degradation product; an amino acid; or a protein degradation product. In some embodiments, the oncotic pressure agent may be selected from low molecular weight dextran, hydroxyethyl starch, modified gelatin, and recombinant albumin.

Representative electrolytes that may be used in the oxygen transporting formulations described herein include, but are not limited to: sodium salts such as sodium chloride, sodium hydrogen carbonate, sodium citrate, sodium lactate, sodium sulfate, sodium dihydrogen phosphate, disodium hydrogen phosphate, sodium acetate, sodium glycerophosphate, sodium carbonate, an amino acid sodium salt, sodium propionate, sodium β-hydroxybutyrate, and sodium gluconate; potassium salts such as potassium chloride, potassium acetate, potassium gluconate, potassium hydrogen carbonate, potassium glycerophosphate, potassium sulfate, potassium lactate, potassium iodide, potassium dihydrogen phosphate, dipotassium hydrogen phosphate, potassium citrate, an amino acid potassium salt, potassium propionate, and potassium β-hydroxybutyrate; calcium salts such as calcium chloride, calcium gluconate, calcium lactate, calcium glycerophosphate, calcium pantothenate, and calcium acetate; magnesium salts such as magnesium chloride, magnesium sulfate, magnesium glycerophosphate, magnesium acetate, magnesium lactate, and an amino acid magnesium salt; ammonium salts such as ammonium chloride; zinc salts such as zinc sulfate, zinc chloride, zinc gluconate, zinc lactate, and zinc acetate; iron salts such as iron sulfate, iron chloride, and iron gluconate; copper salts such as copper sulfate, and manganese salts such as manganese sulfate. In some embodiments, the electrolyte may be selected from sodium chloride, potassium chloride, magnesium chloride, disodium hydrogen phosphate, dipotassium hydrogen phosphate, potassium dihydrogen phosphate, sodium lactate, sodium acetate, sodium citrate, potassium acetate, potassium glycerophosphate, calcium gluconate, calcium chloride, magnesium sulfate, and zinc sulfate.

Representative saccharides that may be used in the oxygen transporting formulations described herein include, but are not limited to, glucose, fructose, xylitol, sorbitol, mannitol, dextrin, glycerin, sucrose, trehalose, glycerol, maltose, lactose, and erythritol. In some embodiments, the saccharide may be selected from glucose, fructose, xylitol, sorbitol, mannitol, dextrin, glycerin, and sucrose.

Representative amino acids that may be used in the oxygen transporting formulations described herein include, but are not limited to, lysine, lysine hydrochloride, lysine acetate, asparagine, glutamine, aspartic acid, glutamic acid, serine, threonine, tyrosine, methionine, cystine, cysteine, cysteine hydrochloride, cysteine malate, homocysteine, isoleucine, leucine, phenyalanine, tryptophan, valine, arginine, arginine hydrochloride, histidine, histidine hydrochloride, alanine, proline, and glycine. In some embodiments, the amino acid may be selected from lysine, asparagine, glutamine, aspartic acid, glutamic acid, serine, threonine, tyrosine, methionine, cystine, cysteine, homocysteine, and glycine.

Antioxidants that may be used in the oxygen transporting formulations described herein include, but are not limited to, sodium hydrogen sulfite, sodium sulfite, sodium metabisulfite, rongalite, ascorbic acid, sodium ascorbate, erythorbic acid, sodium erythorbate, cysteine, cysteine hydrochloride, homocysteine, glutathione, thioglycerol, α-thioglycerin, sodium edetate, thioglycolate, sodium pyrosulfite 1,3-butylene glycol, disodium calcium ethylenediaminetetraacetate, disodium ethylenediaminetetraacetate, an amino acid sulfite such as L-lysine sulfite, butylhydroxyanisole, butylhydroxytoluene, propylgallate, ascorbyl palmitate, vitamin E and derivatives thereof such as dl-α-tocopherol, tocopherol acetate, d-δ-tocopheryl, mixed tocopherols, and Trolox, guaiac, nordihydroguaiaretic acid, L-ascorbate stearate esters, soybean lecithin, palmitic acid ascorbic acid, benzotriazole, and pentaerthrityl-tetrakis [3 -(3,5-di-t-butyl-4-hydroxyphenyl)propionate]2-mercaptobenzimidazole. In some embodiments, the antioxidant is selected from sodium hydrogen sulfite, sodium sulfite, ascorbic acid, homocysteine, dl-α-tocopherol, tocopherol acetate, glutathione, and Trolox.

The pH of the oxygen transporting formulation may be adjusted using either an acidic pH adjuster of an alkaline pH adjuster. Examples of acidic pH adjusters include, but are not limited to, adipic acid, sodium caseinate, hydrochloric acid, diluted hydrochloride acid, sulfuric acid, aluminum potassium sulfate, citric acid, sodium dihydrogen citrate, glycine, glucono-δ-lactone, gluconic acid, sodium gluconate, crystal sodium dihydrogen phosphate, succinic acid, acetic acid, ammonium acetate, tartaric acid, D-tartaric acid, lactic acid, glacial acetic acid, monosodium fumarate, sodium propionate, boric acid, ammonium borate, maleic acid, malonic acid, malic acid, anhydrous disodium phosphate, methanesulfonic acid, phosphoric acid, and dihydrogen phosphates such as potassium dihydrogen phosphate and sodium dihydrogen phosphate. In some embodiments, the acidic pH adjuster is selected from hydrochloric acid, citric acid, succinic acid, acetic acid, lactic acid, glacial acetic acid, phosphoric acid, potassium dihydrogen phosphate, and sodium dihydrogen phosphate. Example of alkaline pH adjusters include, but are not limited to, dry sodium carbonate, sodium citrate, sodium acetate, diisopropanolamine, sodium L-tartrate, lactates such as calcium lactate and sodium lactate, borax, sodium maleate, sodium malonate, sodium malate, potassium hydroxide, calcium hydroxide, sodium hydroxide, magnesium hydroxide, sodium hydrogen carbonate, sodium carbonate, triisopropanolamine, monoethanolamine, triethanolamine, anhydrous sodium acetate, anhydrous sodium monohydrogen phosphate, meglumine, phosphates such as trisodium phosphate, sodium salts of barbital, and hydrogen phosphates such as disodium hydrogen phosphate and dipotassium hydrogen phosphate. In some embodiments, the alkaline pH adjuster is selected from sodium acetate, sodium hydroxide, sodium hydrogen carbonate, sodium carbonate, trisodium phosphate, disodium hydrogen phosphate, and dipotassium hydrogen phosphate. In some embodiments, the pH of the oxygen transporting formulation may be adjusted by bubbling carbon dioxide gas through the formulation.

Representative examples of isotonizing agents that may be used in the oxygen transporting formulations described herein include, but are not limited to, aminoethylsulfonic acid, sodium hydrogen sulfite, potassium chloride, calcium chloride, sodium chloride, benzalkonium chloride, magnesium chloride, saccharides such as lactose, concentrated glycerin, glucose, fructose, xylitol, and glycerin, sugar alcohols such as D-sorbitol and D-mannitol, citric acid, sodium citrate, crystal sodium dihydrogen phosphate, calcium bromide, sodium bromide, sodium hydroxide, physiological saline, sodium tartrate dihydrate, sodium hydrogen carbonate, nicotinamide, sodium lactate solutions, propylene glycol, benzyl alcohol, boric acid, borax, anhydrous sodium pyrophosphate, phosphoric acid, disodium hydrogen phosphate, potassium dihydrogen phosphate, sodium dihydrogen phosphate, and macrogol 4000. In some embodiments, the isotonizing agent may be selected from potassium chloride, sodium chloride, concentrated glycerin, disodium hydrogen phosphate, and potassium dihydrogen phosphate.

Formulations suitable for administration include, for example, aqueous sterile injections suspensions, which can contain antioxidants, buffers, bacteriostats, and solutes that render the formulation isotonic with the blood of the intended recipient; and aqueous and non-aqueous sterile suspensions, which can include suspending agents and thickening agents. The formulations can be presented in unit-dose and multi-dose contains, for example sealed ampoules and vials, and can be stored in a free dried (lyophilized) condition requiring only the condition of the sterile liquid carrier, for example, water for injections, prior to use. Extemporaneous injection suspensions can be prepared a sterile powder, etc. It shall be understood that in addition to the ingredients particularly mentioned above, the compositions can include other agents conventional in the art having regard to the type of formulation in question.

The compositions disclosed herein can be administered intravenously, intramuscularly, or intraperitoneally by infusion or injection. Dispersions of the particles can be prepared in water or alternatively in glycerol, liquid polyethylene glycols, triacetin, and mixtures thereof. Under ordinary conditions of storage and use, the preparations can contain a preservative to prevent the growth of microorganisms.

The formulations suitable for injection or infusion can include sterile aqueous dispersion or suspensions or sterile powders comprising the particles, which are adapted for extemporaneous preparation of sterile injectable or infusible dispersions or suspensions. The ultimate dosage form should be sterile, fluid, and stable under the conditions of manufacture or storage. The liquid carrier or vehicle can be a solvent or liquid dispersion medium including, for example, water, ethanol, a polyol (for example, glycerol, propylene glycol, liquid polyethylene glycols, and the like), vegetable oils, nontoxic glyceryl esters, and suitable mixtures thereof. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Sterile injectable dispersions or suspensions are prepared by incorporating the particles described herein in the required amount in the appropriate solvent with various other ingredients enumerated herein, as required, followed by filter sterilization. Alternatively, the particles may be prepared as sterile powders using vacuum drying and freeze drying techniques that may be later combined with sterile-filtered solutions.

The oxygen transporting formulation as described herein can be used in any application where oxygen carriers are required. For example, the formulation may be administered to a living organism, for example or a human or other mammal, for supplying oxygen to an ischemic site or a tumor tissue or for blood infusion when bleeding occurs. Alternatively, the composition may be used as an organ storage perfusion solution, an extracorporeal circulation solution, or as a cell culture solution. The solutions described herein may be injected intravenously to a patient who, for whatever reason, is deprived of oxygen in its tissues and/or organs.

By way of non-limiting illustration, examples of certain embodiments of the present disclosure are provided below.

### EXAMPLES

The follow examples are set forth to illustrate the compositions and methods according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

### Example 1

### Preparation of Nanoparticles from Bovine Hemoglobin Using an Oxidized Dextran Crosslinker

Purification of Hemoglobin

Bovine red blood cells (Quad Five, Ryegate, Mont.) were separated from whole blood by centrifugation at 1100 g. The supernatant of serum and buffy coat were removed via aspiration.

The remaining red blood cells were washed three times with 0.9 wt. % sodium chloride via centrifugation at 3700 g. Cells were then lysed by the addition of an equal volume of 3.75 mM phosphate buffer (PB) solution for 1 hour at 4° C. Bovine hemoglobin (bHb) was ultrapurified on a series of polyethersulfone tangential flow filtration membranes of 50 to 500 kDa as described in Palmer et al., Tangential flow filtration of hemoglobin. Biotechnol. Prog. 2009, 25, 189-199. Purified bHb was concentration to at least 200 mg/mL and stored in PB at −80 ° C.

### Preparation of Oxidized Dextran

Dextran-40 (Sigma Aldrich, St. Louis, Mo.), a polymer of glucose rings, was prepared as a 10% solution in deionized (DI) water. Sodium meta periodate (Fisher Scientific, Pittsburgh, Pa.) was added at a 1:1 molar ratio of periodate to glucose subunits and reacted for one hour at 22° C. in the dark. The oxidized dextran product (OD-40) was dialyzed into DI water over 3 days at 4° C. using an 8 kDa cellulose membrane and stored at 4° C.

### Synthesis of Nanoparticles

One volume of bHb solution was prepared as a 15 mg/mL solution in DI water. The solution temperature was controlled with an ice bath on top of a stir plate and maintained below 4° C. during all steps unless otherwise noted. Under good mixing conditions, 1.5 volumes of 100% ethanol at 4° C. were added drop-wise over 1 min. After ethanol addition was complete and the mixture began to appear turbid, it was left to react without stirring for 3 minutes. Once particle formation was complete, OD-40 was added drop-wise under gentle mixing until reaching a concentration of 2 wt %. The surface stabilization reaction continued for 60 min without stirring. Excess sodium cyanoborohydride (Fisher Scientific) was added to a 0.1 M concentration, and the suspension was held for 30 min. The remaining chemical reagents were removed with a TFF on a 50 nm membrane. The particles were washed into DI water with 10 volume exchanges using constant volume diafiltration. A further 5 volume exchanges were performed to exchange particles into the desired buffer, in this case phosphate buffered saline (PBS). The same TFF system, operated without a buffer feed line, was used to concentrate the final particle suspension back to the volume of the original bHb solution.

### Analysis

Particle size was estimated using dynamic light scattering (DLS; Brookhaven Instruments, Holtsville, N.Y.) using the NNLS fit algorithm. Size and zeta potential were measured by tunable resistive pulse sensing using the qNano device (Izon Science, Medford, Mass.). Scanning electron microscopy (SEM) was performed on samples which were desalted into DI water using the Apreo device (FEI, Hillsboro, Oreg.), with settings indicated on each micrograph.

### Example 2

### Preparation of Nanoparticles from Human Hemoglobin and Serum Albumin Using a Glutaraldehyde Crosslinker

### Purification of Hemoglobin

Outdated human red blood cells (Wexner Medical Center, Columbus OH) were washed with normal saline three times via centrifugation at 3700 g to remove storage media. Cells were then lysed by the addition of an equal volume of 3.75 mM PB for 1 hour at 4 ° C. Human hemoglobin (hHb) was ultrapurified and concentrated using TFF as described in Example 1.

### Synthesis of Nanoparticles

A protein mixture of 75% hHb and 25% HSA (Octapharma, Lachen, Switzerland) was prepared at a total protein concentration of 15 mg/mL in DI water. The solution was held at 4° C. during all steps. Under good mixing conditions, 1.5 volumes of 100% ethanol at 4° C. were added drop-wise over 1 min. After ethanol addition was complete and the mixture began to appear turbid, it was left to react without stirring for 5 minutes. Once particle formation was complete, glutaraldehyde (Sigma Aldrich) was added drop-wise as a 10% solution under gentle mixing until reaching a concentration of 5 mM glutaraldehyde. The surface stabilization reaction continued for 60 min without stirring. Excess sodium cyanoborohydride (Fisher Scientific) was added to a 0.1 M concentration, and the suspension was held for 30 min. The remaining chemical reagents were removed with a TFF on a 50 nm membrane. The particles were washed into DI water with 10 volume exchanges using constant volume diafiltration and prepared for analysis. Analysis was performed using identical methods to those described in Example 1.

### Example 3

### Preparation of Nanoparticles from Bovine Hemoglobin Using a Glutaraldehyde Crosslinker

### Purification of Hemoglobin

Bovine Hemoglobin (bHb) was prepared by a similar procedure to that described in Example 1.

### Synthesis of Nanoparticles

Initial formation of particles was performed by addition of ethanol to a solution of bHb as described in Example 1. After waiting 3 minutes after the mixture became turbid, glutaraldehyde (Sigma Aldrich) was added drop-wise as a 10% solution under gentle mixing to a total concentration of 5 mM. Subsequent steps including reaction with sodium cyanoborohydride and purification by TFF were identical to Example 1.

### Example 4

### Preparation of Serum Albumin-Coated Hemoglobin Nanoparticles from Human Hemoglobin Using a Glutaraldehyde Crosslinker

### Purification of Hemoglobin

Human hemoglobin (hHb) was prepare by a similar procedure to that described in Example 2.

### Synthesis of Nanoparticles

One volume of hHb solution was prepared as a 15 mg/mL solution in DI water. The solution temperature was controlled with an ice bath on top of a stir plate and maintained below 4° C. during all steps unless otherwise noted. Under good mixing conditions, 1.5 volumes of 100% ethanol at 4° C. were added drop-wise over 1 min. After ethanol addition was complete and the mixture began to appear turbid, it was left to react without stirring for 3 minutes. To coat the particles, a concentrated stock of 250 mg/mL HSA (Octapharma, Lachen, Switzerland) was added drop-wise to reach relative protein concentrations of 90% hHb and 10% HSA. The particles were stabilized with glutaraldehyde (Sigma Aldrich) which was added drop-wise as a 10% solution under gentle mixing until reaching a concentration of 5 mM glutaraldehyde. The surface stabilization reaction continued for 60 min without stirred. Excess sodium cyanoborohydride (Fisher Scientific) was added to a 0.1 M concentration, and the suspension was held for 30 min. The remaining chemical reagents were removed with a TFF on a 50 nm membrane. The particles were washed into DI water with 10 volume exchanges using constant volume diafiltration and prepared for analysis.

### Example 5

### Comparative Analysis of Prepared Hemoglobin Nanoparticles

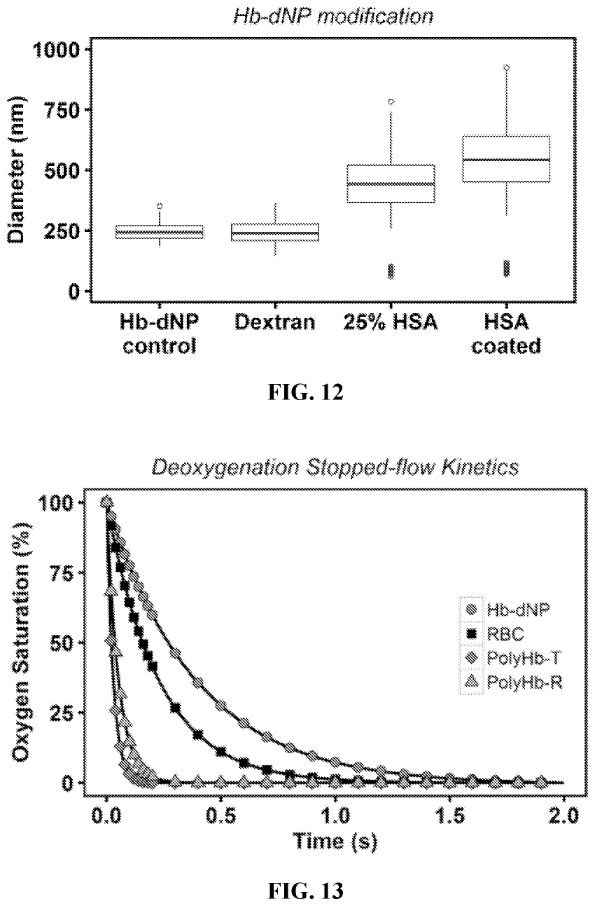

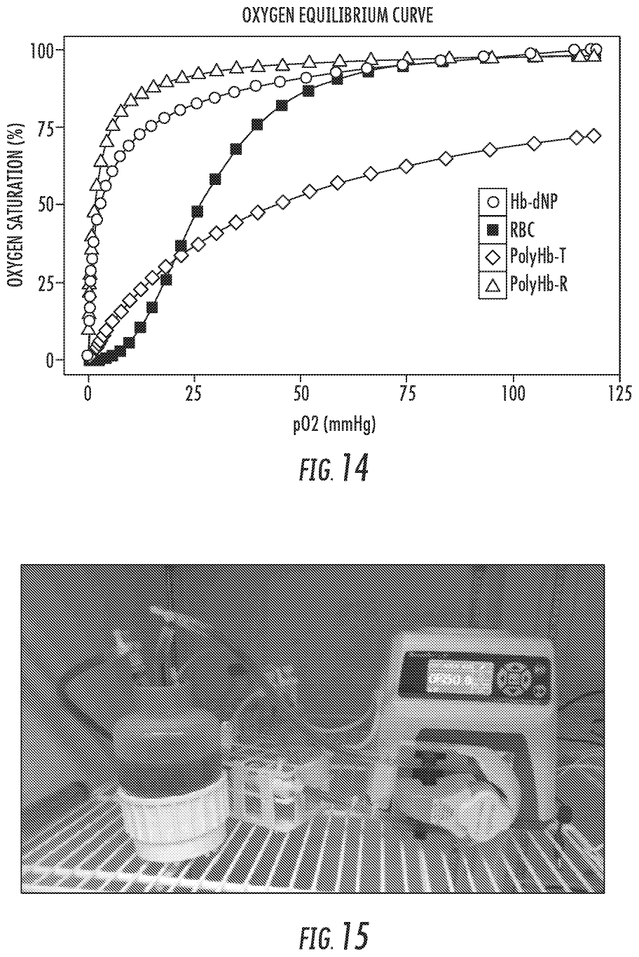

FIG. 11 provides the measured zeta potential and FIG. 12 the provides the measured size for hemoglobin nanoparticles crosslinked with glutaraldehyde (Hb-dNP), hemoglobin nanoparticles crosslinked with oxidized dextran (Dextran), 75% Hb-25% HSA nanoparticles (25% HSA), or HSA-coated nanoparticles (HSA coated) based on the analyses performed as described in Examples 1-4. The dextran crosslink coating was found to increase the stability of the hemoglobin nanoparticles without increasing particle size.

### Example 6

### Comparison of Oxygen Binding of Hemoglobin Nanoparticles With Other Artificial Oxygen Carriers

The hemoglobin nanoparticles used herein are prepared as described in Example 3. Relaxed state (R) polymerized hemoglobin (PolyHb-R) and tense state (T) polymerized hemoglobin (PolyHb-T) were both prepared according to the procedure described in Belcher, D. A. et al. Sci. Rep. 2020, 10:11372. Data for red blood cells (RBC) used in this analysis were referenced from Coin, J. T., and Olson, J. S., J. Biol. Chem. 1979, 254:1178-1190.

The kinetics of O₂ offloading (k_{off,O2}) for the hemoglobin nanoparticles (Hb-dNP), red blood cells (RBC), tense state (T) polymerized hemoglobin (PolyHb-T), and relaxed state (R) polymerized hemoglobin (PolyHb-R) were measured with an Applied Photophysics SF-17 microvolume stopped-flow spectrophotometer (Applied Photophysics Ltd., Surrey, United Kingdom) using protocols previously described, see Rameez, S. and Palmer, A. F. Langmuir, 2011, 27:8829-8840; and Rameez, S. et al. Biotechnol Prog. 2012, 28:636-645. The data obtained are provided in FIG. 13 and Table 1.

Oxygen equilibrium curves as found in FIG. 14 for Hb-dNP, PolyHb-T, and PolyHb-R were measured using a Hemox™ Analyzer (TCS Scientific Corp., New Hope, Pa., USA) at 37 ° C. using the procedure described in Palmer, A. F., Sun, G., and Harris, D. R. Biotechnol Prog. 2009, 25(1):189-199. The oxygen equilibrium curves were fit to a Hill model as described to provide the cooperativity coefficient (n) and P₅₀ for each of Hb-dNP, RBC, PolyHb-T, and PolyHb-R.

TABLE 1
Comparative Oxygen Binding Data

| | koff (s⁻¹) at 21° C. | n (Hill coefficient) | Pso (mm Hg) |
|---|---|---|---|
| Hb-dNP | 2.56 | 1.3 | 7.72 |
| RBC | 4.04 | 2.8 | 26.5 |
| PolyHb-T | 34.17 | 0.83 | 43.9 |
| PolyHb-R | 19.20 | 1.1 | 1.60 |

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

Unless defined otherwise, all technical and scientific terms used herein have the same meanings as commonly understood by one of skill in the art to which this disclosure belongs. Publications cited herein the materials for which they are cited are specifically incorporated by reference.

1. An oxygen transporting formulation comprising a plurality of particles;
wherein the plurality of particles has an average particle size of less than 1000 nm diameter;
wherein each particle within the plurality of particles comprises at least 25% by weight hemoglobin and optionally other proteins;
wherein each particle has an outer surface;
wherein the hemoglobin and/or other proteins present on the outer surface has been substantially crosslinked using a chemical crosslinker; and
wherein the oxygen transporting formulation is sufficiently free of surfactant.

2. The oxygen transporting formulation of claim 1, wherein each particle is substantially composed of hemoglobin of human origin or hemoglobin of bovine origin; or
wherein each particle is composed of a mixture of hemoglobin and human serum albumin (HSA).

3. (canceled)

4. (canceled)

5. The oxygen transporting formulation of claim 4, wherein each particle within the plurality of particles comprises at least 25%, at least 30%, at least 40%, at least 50%, at least 75%, at least 80%, at least 90%, at least 95%, or more by weight hemoglobin based on the total weight of all proteins present in the particles.

6. (canceled)

7. (canceled)

8. The oxygen transporting formulation of claim 1, wherein the plurality of particles is characterized in having an average particle size from 100 to 1000 nm, from 100 to 900 nm, from 100 to 800 nm, from 100 to 700 nm, from 100 to 600 nm, from 100 to 500 nm, from 100 to 400 nm, or from 100 to 300 nm.

9. The oxygen transporting formulation of claim 1, wherein the plurality of particles is characterized in having a polydispersity index (PDI) from 0 to 0.3 or from 0 to 0.1.

10. (canceled)

11. (canceled)

12. The oxygen transporting formulation of claim 1, wherein the plurality of particles is character is having a zeta potential from −40 mV to −10 mV, from −40 mV to −20 mV, from −40 mV to −30 mV, from −35 mV to −15 mV, from −35 mV to −20 mV, from −35 mV to −25 mV, or from −35 mV to −30 mV.

13. The oxygen transporting formulation of claim 1, wherein the chemical crosslinker is an aldehyde-containing chemical crosslinker, or wherein chemical crosslinker is glutaraldehyde or oxidized dextran.

14. (canceled)

15. (canceled)

16. The oxygen transporting formulation of claim 1, wherein each particle within the plurality of particles has been further surface treated with one or more surface modulators.

17. The oxygen transporting formulation of claim 16, wherein the one or more surface modulators is selected from human serum albumin (HSA), a polysaccharide such as dextran, a polyelectrolyte, or one or more red blood cell membrane components.

18. The oxygen transporting formulation of claim 1, wherein the oxygen transporting formulation further comprises at least one reducing agent.

19. The oxygen transporting formulation of claim 18, wherein the at least one reducing agent is selected from N-acetyl-L-cysteine, ascorbic acid, methylene blue, or mixtures thereof.

20. A process for the synthesis of an oxygen transporting formulation of claim 1 comprising:
(a) adding a desolvating agent to an aqueous solution of hemoglobin and optionally other proteins to provide a plurality of particles;
(b) adding a chemical crosslinker to substantially crosslink the hemoglobin and/or other optional proteins on the outer surface of each particle within the plurality of particles formed in step (a);
(c) isolating the plurality of particles provided from step (b) by substantially removing excess desolvating agent, chemical crosslinker, solvent, and other by-products; and
(d) resuspending the plurality of particles isolated in step (c) in a pharmaceutically acceptable carrier.

21. The process of claim 20, wherein the desolvating agent comprises a water-miscible polar solvent wherein which hemoglobin and/or the other optional proteins are insoluble.

22. The process of claim 20, wherein the desolvating agent is selected from ethanol, methanol, acetone, isopropyl alcohol, or combinations thereof.

23. The process of claim 20, wherein the process further comprises:
(b1) adding a reducing agent after addition of the chemical crosslinker in step (b) to further stabilizing the substantial crosslinking on the outer surface of each particle and deactivate any residual chemical crosslinker.

24. The process of claim 23, wherein the reducing agent may be selected from sodium borohydride or sodium cyanoborohydride.

25. The process of claim 23, wherein the reducing agent is added to reduce imine crosslink intermediates on the surface of the particle when an aldehyde-containing crosslinker is used; or
wherein the reducing agent is added to reduce imine intermediates in solution formed by the reaction of excess aldehyde-containing crosslinker and an amine additive.

26. (canceled)

27. The process of claim 25, wherein the amine additive may be selected from Tris buffer or glycine.

28. The process of claim 20, wherein the process comprises isolating the plurality of particles in step (c) by centrifugation or tangential flow filtration (TFF).

29. The process of claim 20, wherein the process comprises resuspending the plurality of particles in step (d) in an injectable solution suitable for clinical use.
